(19) United States
(12) Patent Application Publication
(10) Pub. No.: US 2021/0154379 A1
(43) Pub. Date: May 27, 2021

(54) HAND TOOL WITH INTEGRATED MICROPUMP AND DRUG RESERVOIR FOR INTRACOCHLEAR DRUG DELIVERY

(71) Applicant: Charles Stark Draper Laboratory, Inc., Cambridge, MA (US)

(72) Inventors: Vishal Tandon, Somerville, MA (US); Ernest Kim, Cambridge, MA (US); Jeffrey Borenstein, West Roxbury, MA (US)

(21) Appl. No.: 17/100,309

(22) Filed: Nov. 20, 2020

Related U.S. Application Data
(60) Provisional application No. 62/938,549, filed on Nov. 21, 2019.

(51) Int. Cl. A61M 1/00; A61M 39/24

(52) U.S. Cl. CPC A61M 1/0064; A61M 1/009; A61M 1/0084; A61M 1/0082; A61M 2210/0668; A61M 39/24; A61M 2207/10; A61M 2205/0205; A61M 1/0086

(57) ABSTRACT

The present disclosure provides a handpiece for trans-canal delivery of a therapeutic substance to the inner ear. The handpiece can be inserted into the middle ear via a surgical tympanotomy approach. The handpiece can be integrated with a micropump and a fluid reservoir. The handpiece can enable a controlled injection of a therapeutic substance directly through the round window membrane and into the inner ear. The direct delivery of the therapeutic substance to the inner ear can enable the delivery of a precise amount of therapeutic substance into the inner ear. The micropump can include a self-contained fluid reservoir that can provide predetermined volumes of fluid to precise areas of the patient.

### CROSS REFERENCE TO RELATED APPLICATIONS

The present application claims priority under 35 U.S.C. § 119(e) to U.S. Provisional Patent Application No. 62/938,549, titled “HAND TOOL WITH INTEGRATED MICROPUMP AND DRUG RESERVOIR FOR INTRACOCHLEAR DRUG DELIVERY,” filed Nov. 21, 2019, which is incorporated herein in its entirety by reference.

### BACKGROUND

Delivery of therapeutics to the human inner ear can be challenging for clinicians. There are two anatomic “windows” from the middle ear to the inner ear, which are referred to as the oval window and the round window. Each of these windows can include a semi-permeable membrane. Drug delivery to the inner ear can occur when a therapeutic substance crosses at least one of these membranes.

### SUMMARY

Inner ear drug delivery can use diffusion to cross one or both of the membranes of the anatomic windows to the inner ear. Relying on diffusion across a membrane poses a number of difficulties. For example, diffusing therapeutic substances across the membranes can introduce a lack of precision in terms of dose delivery. Relying on diffusion can also limit the size and characteristics of the molecules of a therapeutic substance because, for example, not all substances can diffuse across the membranes. Another example challenge is that the round window membrane permeability can vary between patients or during states of inflammation. This disclosure describes a handpiece that can overcome these technical challenges by delivering a therapeutic substance directly to the inner ear.

Penetration of the round window membrane can enable delivery of a therapeutic substance such as a drug directly into cochlear fluids using a pump, but placement of a catheter can be a delicate process, and such penetrations can foul or close quickly. Furthermore, standard pumps may be bulky and tubing connections between a pump and the catheter can result in a large dead volume. This handpiece provided in this disclosure can include an integrated micropump to facilitate trans-round window membrane delivery of therapeutic substance. The micropump can be small, portable, and self-contained so that the entire apparatus can be easily held in a hand and manipulated with fine control. For example, the handpiece can include an integrated fluid reservoir and an integrated micropump, thereby reducing dead volume and increasing maneuverability of the handpiece.

At least one aspect of the present solution is directed to handpiece device that can deliver a fluid into an inner ear. The handpiece device can include a shaft. The shaft can include a first fluidic channel. The handpiece device can include a tip portion coupled with a first end of the shaft. The tip portion can include an outlet and a second fluidic channel in fluid communication with the first fluidic channel. The handpiece device can include a collar coupled with the tip portion a predetermined distance from the outlet. The collar can seat with an anatomic structure, such as a round window membrane or an oval window, and control a distance that the tip portion can project into a cochlea when the tip portion is inserted into an ear canal. The shaft of the handpiece device can be integrated with a micropump and a fluid reservoir. The micropump can pump a fluid from the fluid reservoir to the outlet via the first fluidic channel.

In some implementations, the handpiece device can include an angled portion coupling the tip portion to the shaft. In some implementations, the angled portion can have a third fluidic channel in fluid communication with the first fluidic channel and the second fluidic channel. In some implementations, at least one of the angled portion or the tip portion are separable from the shaft. In some implementations, at least one of the angled portion, the tip portion, or the shaft and can be coupled together using one or more of a snap-on connector, a friction fit connection, a press-fit connection, a knurled nut, or a Luer lock connection. In some implementations, at least one of the angled portion or the tip portion can rotate, while coupled to the shaft, around an axis parallel to a length of the shaft.

In some implementations, the angled portion of the handpiece device forms an angle between the shaft and the tip portion between 90 degrees and 170 degrees. In some implementations, the angled portion, the tip portion, and the shaft are manufactured from a single contiguous piece of material. In some implementations, the shaft or the tip portion are manufactured from one or more of stainless steel or a sterilizable plastic. In some implementations, the shaft of the handpiece device can include a first plurality of fluidic channels. In some implementations, the tip portion can include a second plurality of fluidic channels. In some implementations, each of the second plurality of fluidic channels are in fluidic communication with a respective one of the first plurality of fluidic channels.

In some implementations, the outlet of the tip portion includes a needle portion extending past the collar portion. In some implementations, the needle portion can pierce an anatomic structure, such as the oval window or the round window separating the middle ear of a patient from the inner ear of the patient. In some implementations, the needle portion can extend past the collar portion by a distance between 1 millimeter and 4 millimeters, or by a distance between 2 millimeters and 3 millimeters. In some implementations, the needle portion can have a gauge size between 25 and 30. In some implementations, the shaft portion can have a diameter of 4 millimeters, 5 millimeters, or 6 millimeters. In some implementations, the shaft portion can have a length between 90 millimeters and 160 millimeters.

In some implementations, the outlet of the tip portion can be positioned at a distal end of the tip portion. In some implementations, the distal end can form an angle between the outlet and the tip portion. In some implementations, the angle can be between 70 degrees and 170 degrees. In some implementations, the angle can be between 75 degrees and 130 degrees. In some implementations, the angle can be between 90 degrees and 120 degrees. In some implementations, the angle can be or between 110 degrees and 120 degrees. In some implementations, the micropump integrated with the shaft of the handpiece device can include the fluid reservoir, an electromagnetic actuator, a battery, and a control circuit.

At least one aspect of the present disclosure is directed to a system. The system can include a shaft of a handpiece device. The shaft of the handpiece device can define a channel. The system can include a micropump. The micropump can be integrated with the shaft of the handpiece device. The micropump can include a fluid reservoir. The fluid reservoir can include a reservoir inlet and a reservoir outlet. The micropump can include a fluidic channel fluidly coupled to the reservoir outlet of the fluid reservoir. The micropump can include a pump. The pump can include an electromagnetic actuator. The pump can be fluidly connected to the reservoir outlet of the fluid reservoir via the fluidic channel. The pump can include an intake valve. The intake valve can be coupled to the fluidic channel between the fluid reservoir and the pump. The pump, when actuated, can cause fluid in the fluid reservoir to flow to an outlet that is fluidly connected to the channel defined by the shaft of the handpiece. The system can include a tip portion. The tip portion can be coupled to the shaft of the handpiece. The tip portion can include a second channel that receives fluid from the micropump via the channel defined by the shaft.

In some implementations, the micropump can include a plurality of layers. Each of the plurality of layers can define at least one of the fluid reservoir, the fluidic channels, or the outlet. In some implementations, the outlet of the micropump can be fluidly connected to the reservoir inlet of the fluid reservoir via a second valve. In some implementations, the micropump can include one or more fluid capacitors disposed between the fluid reservoir and the pump, or between the pump and the outlet.

At least one aspect of the present disclosure is directed to a method. The method can include providing a handpiece device. The hand piece device can include a shaft comprising a first fluidic channel. The handpiece device can include a tip portion coupled with a first end of the shaft. The tip portion can include an outlet and a second fluidic channel in fluid communication with the first fluidic channel. The handpiece device can include a collar coupled with the tip portion a predetermined distance from the outlet. The collar can seat with an anatomic structure, such as a round window or an oval window of a patient. The collar can control a distance that the tip portion can project into a cochlea when the tip portion is inserted into an ear canal. The shaft can be integrated with micropump and a fluid reservoir. The micropump can pump a fluid from the fluid reservoir to the outlet via the first fluidic channel. The method can include piercing an anatomic membrane covering the anatomic structure of a patient with the tip portion of the handpiece device. The method can include flowing a fluid, using the micropump, through the outlet and into cochlea via the first fluidic channel and the second fluidic channel.

In some implementations, the method can include inserting the tip portion of the handpiece through an ear canal of the patient. In some implementations, the method can include pressing the tip portion through the anatomic membrane in a middle ear of the patient. In some implementations, the method can include seating the collar with the anatomic structure covered by the anatomic membrane in the middle ear, causing a part of the tip portion to extend into the cochlea of the patient. In some implementations, the method can include forming a ventilation hole in a stapes footplate of the patient.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. The foregoing and other objects, aspects, features, and advantages of the disclosure will become more apparent and better understood by referring to the following description taken in conjunction with the accompanying drawings, in which:

FIG. 1 illustrates an example handpiece delivering fluid to the inner ear of a patient, in accordance with one or more implementations;

FIG. 2 illustrates a side view of the example handpiece illustrated in FIG. 1, in accordance with one or more implementations;

FIG. 3 illustrates a cross-sectional view of the example handpiece illustrated in FIG. 1, in accordance with one or more implementations;

FIG. 4 illustrates a side view of the example handpiece illustrated in FIG. 1, in accordance with one or more implementations;

FIG. 5 illustrates an example tip portion for the handpiece illustrated in FIG. 1, in accordance with one or more implementations;

FIG. 6 illustrates an example handpiece with a compression fitting, in accordance with one or more implementations;

FIG. 7 illustrates an enlarged view of the tip of the example handpiece illustrated in FIG. 1, in accordance with one or more implementations;

FIGS. 8A and 8B illustrates the tip of the example handpiece inserted into a round window, in accordance with one or more implementations;

FIGS. 9 and 10 illustrate example fluid reservoirs coupled with the example handpiece illustrated in FIG. 1, in accordance with one or more implementations;

FIGS. 11A, 11B, 11C, and 11D illustrate various views of an example handpiece integrated with a micropump, in accordance with one or more implementations;

FIG. 12 illustrates a top view of an example micropump for use in the example handpiece illustrated in FIGS. 9, 10, and 11A, in accordance with one or more implementations;

FIGS. 13A, 13B, 13C, and 13D illustrate various views of components of an example wearable device 1300 for administering a drug, in accordance with one or more implementations;

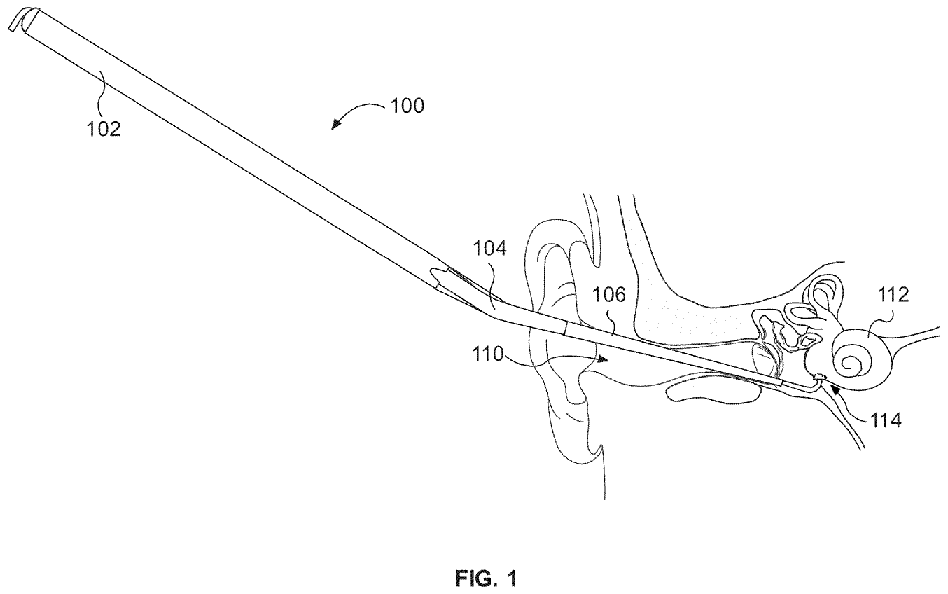

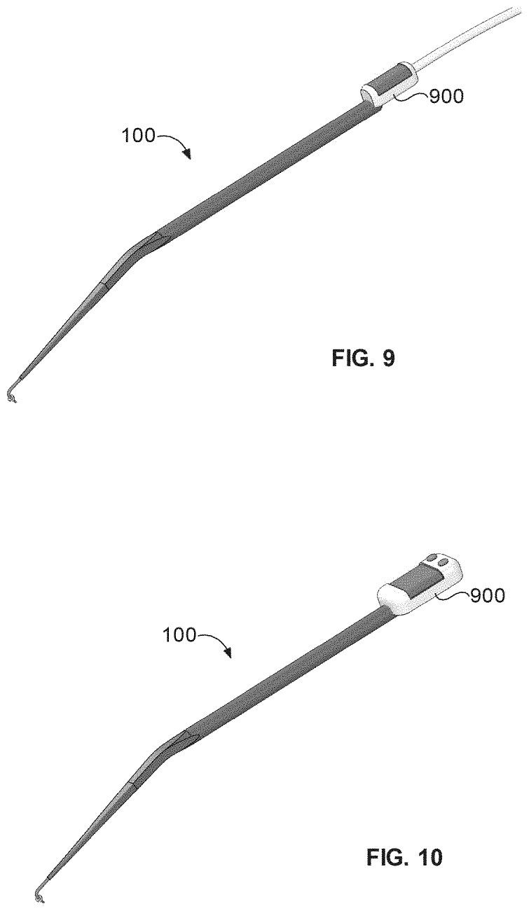

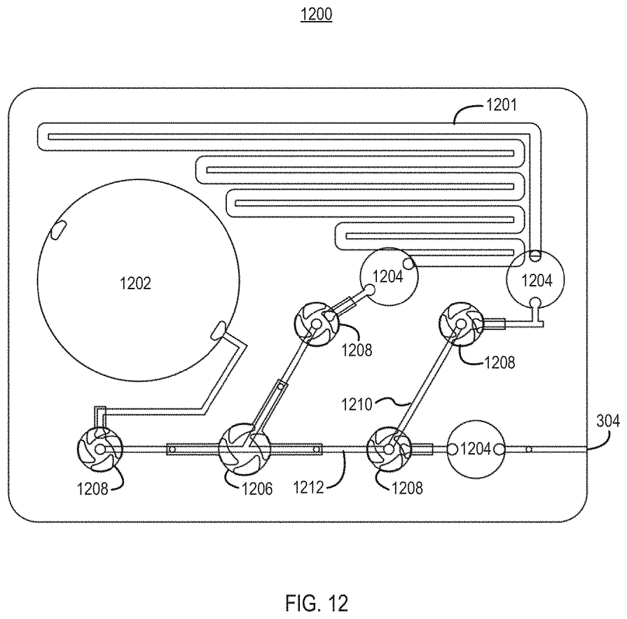

FIG. 12 depicts the example handpiece of FIG. 1 together with the example cannula of FIG. 9 in an arrangement that can be used to facilitate seating the cannula within the round window membrane of a patient, in accordance with one or more implementations; and

FIG. 14 illustrates a block diagram of an example method to flow a fluid into the cochlea using a handpiece, in accordance with one or more implementations.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure provides a handpiece for transcanal delivery of a therapeutic substance to the inner ear. The handpiece can be inserted into the middle ear via a surgical tympanotomy approach. The handpiece can enable a finely controlled injection of a therapeutic substance, such as a drug, directly through the round window membrane and into the inner ear. The direct delivery of the therapeutic substance to the inner ear can enable the delivery of a precise amount of therapeutic substance into the inner ear. Because the therapeutic substance is delivery directly to the inner ear, the delivery of the therapeutic substance is not subject to limitations on molecule size and inconsistent diffusion rates that are present when therapeutic substances are instead diffused across the round window membrane

The handpiece can be or can include an angled device that can be used in the operating room. Following surgical exposure of the round window membrane, the handpiece can be inserted through the ear canal, and can have an appropriate angulation to reach the round window membrane. The handpiece can have an attached tubing system for drug delivery, which can culminate in a fine needle that extends through the center of a collar of the handpiece and directly pierces the round window membrane. In some implementations, the tubing connection can include one or more tubes in parallel or other connected configurations, and can incorporate valves and other fluid connectors, depending on the mode of drug delivery that is required.

The handpiece can also include an integrated pump, associated valves, compliant or resistive elements, reservoirs and flow sensors. At least some of these components can be integrated in a monolithic frame of the handpiece. In some implementations, at least some of these components can be configured to snap into the frame of the handpiece in a modular fashion to enable customized configurations. The handpiece can have flexible internal elements that can enable adjustments at the time of surgery in response to observations regarding the local anatomy of the middle ear and inner ear, or in response to data obtained from prior observation (e.g., radiological imaging) of the local anatomy. These and other aspects are described further below.

FIG. 1 illustrates an example handpiece 100 delivering fluid to the inner ear of a patient. The fluid can be any therapeutic substance or therapeutic agent. The handpiece 100 includes a tool shaft 102, an angled portion 104, and a tip portion 106. The tip portion 106 can also include a collar 108. The handpiece 100 is inserted into the ear canal 110 of the patient for the transcanal delivery of fluid to the cochlea 112 via the round window 114. The tip portion 106 can be used to pierce the round window membrane, or another anatomic structure, to enable fluid to be delivered to the cochlea 112.

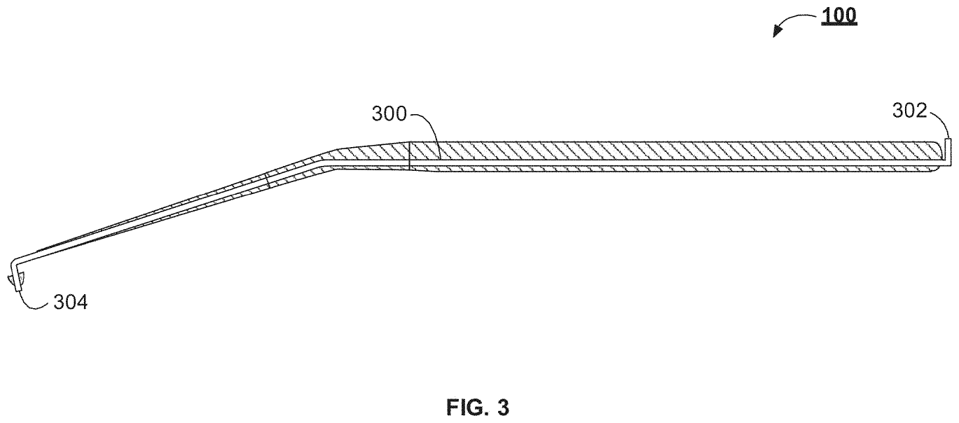

The tool shaft 102 can be held in the hand of a surgeon or by a robotic surgical device. The tool shaft 102 can define a cavity, or channel, through its center. The channel can be, for example, similar to the microfluidic channel 300 described herein below in conjunction with FIG. 3. The tool shaft 102 can be manufactured from a variety of materials, including metals such as aluminum, stainless steel, or other alloys or metals. In some implementations, the tool shaft 102 can be manufactured from one or more plastics or polymers, such as ethylene chlorotrifluoroethylene (ETCFE), ethylene tetrafluoroethylene (ETFE), fluorinated ethylene propylene (FEP), polychlorotrifluoroethylene (PCTFE), polyether ether ketone (PEEK), perfluoroalkoxy alkanes (PFA), polyphenylene sulfide (PPS), polyphenylsulfone (PPSU), or polysulfone (PSU), among others. The tool shaft 102 can be manufactured to be a narrow shaft with a length that is greater than its overall width, to allow the tip portion 106 to be easily positioned within the ear of a patient. However, it should be understood that other configurations of the tool shaft 102 are possible to facilitate positioning of the handpiece within a desired anatomic structure of a patient.

The angled portion 104 can be angled to facilitate positioning the tip portion 106 within an anatomic structure, such as the round window membrane, of the patient. The angled portion 104 can be manufactured as a separate component of the handpiece 100, such that the angled portion 104 can be attached or detached from the tool shaft 102, as needed. When manufactured as separate materials, the angled portion 104 can be coupled to the tool shaft 102 using a type of connector, such as gaskets, O-rings, snap-on connectors, friction-fit connections, press-fit connections, or Luer lock connections, among others. The angled portion 104 can include a second microfluidic channel in communication with the microfluidic channel of the tool shaft 102, such that fluids or a cannula can be transmitted through the channel of the tool shaft 102, through the angled portion 104, and through the tip portion 106 to an outlet of the tip portion 106. In some implementations, the tool shaft 102 and the angled portion 104 can be manufactured as a single contiguous piece of material or combinations of materials, as described herein.

The angle of the angled portion 104 can be selected based on anatomic features of a patient undergoing a procedure using the handpiece 100. For example, different angles of the angled portion 104 may facilitate the positioning of the tip portion 106 within the ear canal 110. The angled portion can be manufactured from a variety of materials, such as aluminum, stainless steel, or other allows or metals. In some implementations, the angled portion 104 can be manufactured from one or more plastics or polymers, such as ethylene chlorotrifluoroethylene (ETCFE), ethylene tetrafluoroethylene (ETFE), fluorinated ethylene propylene (FEP), polychlorotrifluoroethylene (PCTFE), polyether ether ketone (PEEK), perfluoroalkoxy alkanes (PFA), polyphenylene sulfide (PPS), polyphenylsulfone (PPSU), or polysulfone (PSU), among others. The angled portion 104 can be manufactured to be flexible to a certain degree, to allow for the tip portion to better navigate the ear canal 110 of the patient or the middle ear of the patient. In some implementations, the angled portion 104 is not present, and the handpiece 100 instead comprises a tool shaft 102 and tip portion 106.

The tip portion 106 can be manufactured as part of the tool shaft 102 or as part of the angled portion 104. In some implementations, the tip portion 106 can be detachable from one of the tool shaft 102 or the angled portion 104. In some implementations, any combination of the portions of the handpiece can be manufactured as a single piece. For example, the tip portion 106 and the angled portion 104 can be manufactured as a single piece of one or more materials, the tool shaft 102 and the angled portion 104 can be manufactured as a single piece of one or more materials, or the tool shaft 102 and the tip portion 106 can be manufactured as a single piece of one or more materials (e.g., in implementations when the angled portion 104 is not present). The tip portion 106 can be manufactured from a variety of materials, such as aluminum, stainless steel, or other allows or metals. In some implementations, the tip portion 106 can be manufactured from one or more plastics or polymers, such as ethylene chlorotrifluoroethylene (ETCFE), ethylene tetrafluoroethylene (ETFE), fluorinated ethylene propylene (FEP), polychlorotrifluoroethylene (PCTFE), polyether ether ketone (PEEK), perfluoroalkoxy alkanes (PFA), polyphenylene sulfide (PPS), polyphenylsulfone (PPSU), or polysulfone (PSU), among others.

As depicted in FIG. 1, the tip portion 106 can taper along its length to facilitate positioning through the ear canal 110 of the patient and into the middle ear for delivery of a cannula or other fluid. The tip portion 106 can define a microfluidic channel in a central portion of the tip portion 106, such that fluids transmitted through the microfluidic channel of the tool shaft 102 or the angled portion 104 can be transmitted through the tip portion 106 to an outlet of the tip portion 106. The tip portion can include a collar 108, which can be seated around a grooved region in the tip portion 106 or affixed to the tip portion 106 using a glue or another type of adhesive. In some implementations, the collar 108 is manufactured from the same piece of material as the tip portion 106.

The tip portion 106 can have a shape that is configured to seat with an anatomic structure of a patient, such as the round window 114. The tip portion 106 can have an outlet for the microfluidic channels defined within the tool shaft 102, the angled portion 104, and the tip portion 106, each of which can be in communication with one another. The outlet of the tip portion 106 can be disposed on a needle tip portion of the tip portion 106. The needle tip portion of the tip portion 106 can extend into the cochlea 112 of the patient.

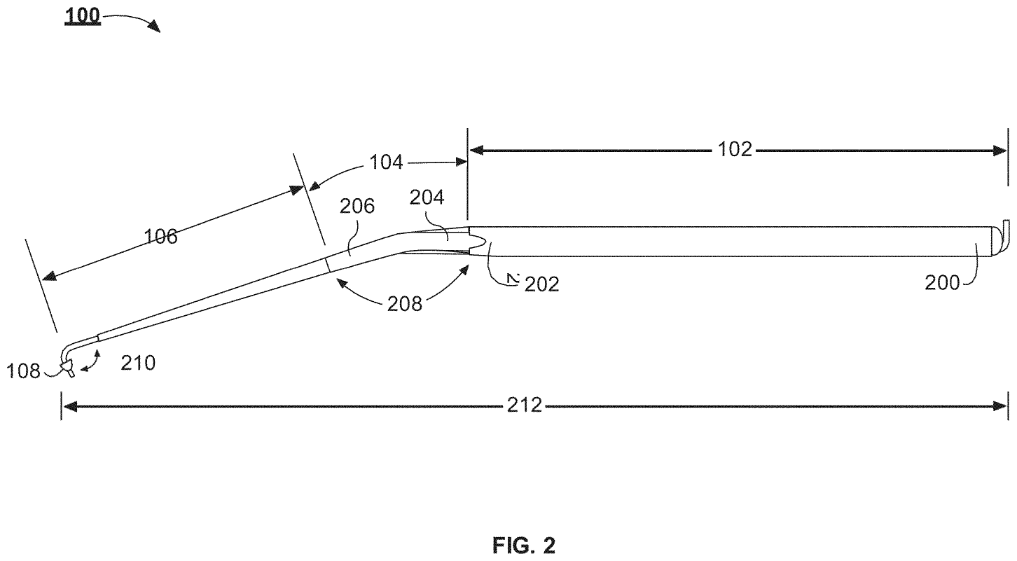

FIG. 2 illustrates a side view of the example handpiece 100. The handpiece 100 includes the tool shaft 102, the angled portion 104, and the tip portion 106. A surgeon can use the tool shaft 102 to hold and manipulate the handpiece 100 and position of the tip portion 106. The outer surface of the tool shaft 102 can include knurling to enable a better grip of the handpiece 100 by the surgeon. In some implementations, one or more portions of the handpiece 100 can be coupled to a surgical robot. In such implementations, the portions of the handpiece 100 can include fasteners or other coupling devices or structures that can couple handpiece to the surgical robot. The tool shaft 102 can include a proximal end 200 and a distal end 202. The tool shaft 102 can have a diameter of about 4 mm, 5 mm, or about 6 mm. The tool shaft 102 can have a length of between about 90 mm and about 150 mm, between about 90 mm and about 130 mm, or between about 100 mm and about 120 mm. In some implementations, the length of the tool shaft 102 is 110 mm.

The distal end of the tool shaft 102 can be coupled with the proximal end 204 of the angled portion 104. The tip portion 106 is coupled with the distal end 206 of the angled portion 104. The angled portion 104 is angled to enable the tip portion 106 to traverse the ear canal (e.g., the ear canal 110 depicted in FIG. 1) in a minimally invasive procedure and reach the round window or another anatomic structure in the ear of a patient. The angled portion 104 forms an angle 208 between the tool shaft 102 and the tip portion 106. The angle 208 can be about 170° and about 90°, between about 170° and about 110°, between about 170° and about 120°, between about 170° and about 140°, or between about 165° and about 155°. The angle 208 can be defined as the angle between a longitudinal axis of the tool shaft 102 and a longitudinal axis of the tip portion 106. The angle 208 is configured to enable transcanal positing of the tip portion 106 at a round window of a patient. The angle 208 can be selected to enable a surgeon to position the tip portion 106 at the round window and provide the surgeon visual access to the ear canal.

The tip portion 106 can be coupled with the distal end 206 of the angled portion 104. The distal portion of the tip portion 106 can be angle. The angle 210 can be between about 70° and about 140°, between about 75° and about 130°, between about 90° and about 120°, between about 100° and about 120°, or between about 110° and about 120°. For example, the angle 210 can be about 105°, 106°, 107°, 108°, 109°, 110°, 111°, 112°, 113°, 114°, 115°, 116°, 117°, 118°, 119°, or 120°. The angle 210 can be selected to position the distal portion of the tip portion 106 substantially perpendicular to the round window when the handpiece 100 is inserted through the ear canal. The angle 210 can be selected based on the anatomical configuration of an inner or a middle ear of the patient. For example, the surgeon can select a handpiece 100 with an appropriate angle 210 based on the position and angle of the round window and the round window niche. In some implementations, the surgeon can determine which angle 210 to select using CT or Mill images of the middle and inner ear. The handpiece 100 can be manufactured with different angle 210 configurations. In some implementations, the surgeon can bend the tip portion 106 to alter the angle 210 during a procedure.

The tip portion 106 can include a collar 108. The collar 108 can be configured to seat within the round window, within an oval window, or within another anatomic structure of a patient. For example, the collar 108 can be made of a semi-flexible material that can conform to the round window in the middle ear of a patient, or conform to a different anatomic structure in the ear of the patient. The collar 108 can be rigid enough to prevent more than a desired portion of the handpiece 100 from extending into the cochlea (e.g., the cochlea 112 depicted in FIG. 1) of a patient. The flexible conformability of the collar 108 can form a seal with one or more anatomic structures of the middle ear of a patient. For example, the collar 108 can seal the round window once the tip portion 106 pierces the round window membrane. The collar 108 can also control the depth the end of the tip portion 106 can be inserted into the cochlea. The collar 108 can include a medical-grade silicone, or another type of semi-flexible or biocompatible material. The collar 108 can be substantially domed or semi-spherical in shape. The diameter of the collar 108, at the widest portion, can be between about 0.5 mm and about 3 mm, between about 0.5 mm and about 2.5 mm, between about 1 mm and about 2 mm, or between about 1.5 mm and about 2 mm.

The handpiece 100 can have an overall length 212 between about 130 mm and about 170 mm, between about 140 mm and about 160 mm, or between about 140 mm and about 150 mm. While described as different portions, the tool shaft 102, the angled portion 104, and the tip portion 106 can each be manufactured as single or multiple pieces. For example, the handpiece 100 can include one, two, or three separate pieces. The handpiece 100 can be separable at the interface between any of the tool shaft 102, the angled portion 104, and the tip portion 106. In some implementations, the interface between the tool shaft 102, the angled portion 104, and the tip portion 106 does not indicate that the portions are separable, such as when one or more of the tool shaft 102, the angled portion 104, or the tip portion 106 are formed from a single contiguous piece of material, or when one or more of the tool shaft 102, the angled portion 104, or the tip portion 106 are coupled together permanently or semi-permanently. For example, the tool shaft 102, the angled portion 104, and the tip portion 106 can be manufactured as a single piece. In other implementations, the angled portion 104 and the tool shaft 102 can form a first piece and the tip portion 106 can form a second piece. In some implementations, the handpiece 100 is reusable. In other implementations, the handpiece 100 is disposable. The handpiece 100 can be manufactured from medically-approved sterilizable materials. For example, the handpiece 100 can be manufactured from 316 stainless steel, or any other type of metal described herein, or a sterilizable plastic or polymer as described herein.

FIG. 3 illustrates a cross-sectional view of the example handpiece 100. The handpiece 100 includes a microfluidic channel 300. The microfluidic channel 300 includes an inlet 302 and an outlet 304. The inlet 302 can be coupled with a reservoir. The reservoir is described further in relation to FIGS. 9 and 10. The microfluidic channel 300 can have a gauge of about 22. The gauge of the microfluidic channel can be between about 12 and 28, between about 16 and about 24, between about 18 and about 22, or between about 20 and 22. The microfluidic channel 300 can have a dead volume of between about 10 μL and about 25 μL, between about 15 μL and about 25 μL, or between about 20 μL and about 25 μL.

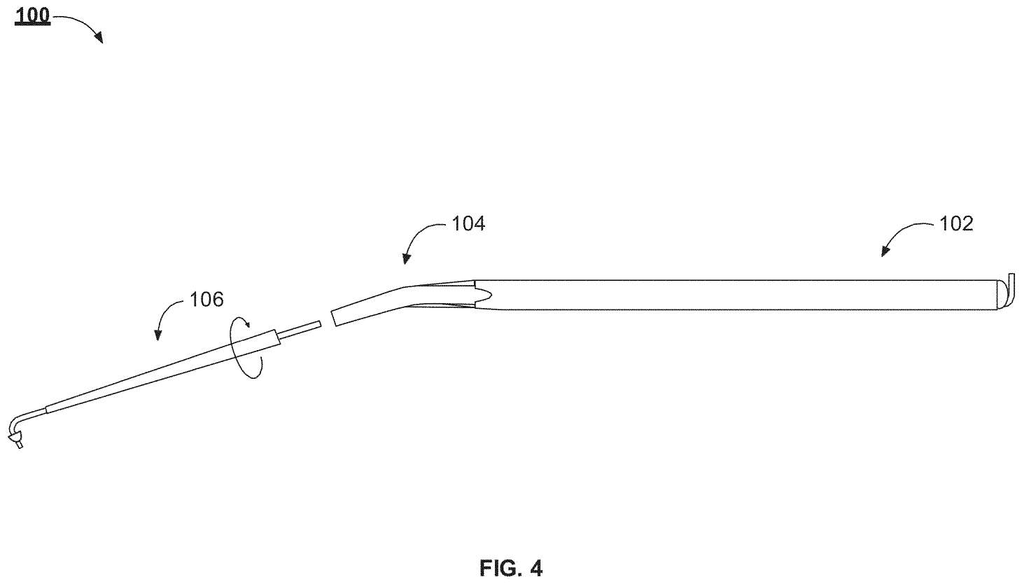

The microfluidic channel 300 can include different portions. For example, each of the tool shaft 102, angled portion 104, and the tip portion 106 can include a different portion of the microfluidic channel 300. The different portions can be a single, continuous channel. In some implementations, the microfluidic channel 300 can separable at the interface between one or more of the portions. In some implementations, the microfluidic channel portions are separable near the interface between the different portions of the handpiece 100. For example, the microfluidic channel portion within the tip portion 106 can extend past the tip portion 106 (as illustrated in FIG. 4) and the microfluidic channel portion within the angled portion 104 can stop prior to the distal end 206, such that portion of the microfluidic channel extending from the tip portion 106 can be received by the angled portion 104. In some implementations, the handpiece 100 can include a plurality of microfluidic channels 300. For example, the handpiece 100 can include different microfluidic channels 300 for delivering different therapeutic agents. In some implementations, a second microfluidic channel 300 can be used to evacuate fluid from the cochlea. The microfluidic channel 300 can be configured to provide or otherwise seat a cannula, such as the cannula 904 described herein in conjunction with FIG. 9, into the cochlea 112 of a patient.

FIG. 4 illustrates a side view of the example handpiece 100. In some implementations, one or more of the portions of the handpiece 100 are separable from one another. FIG. 4 illustrates an example handpiece 100 with a separable tip portion 106. The tip portion 106 can be separated from the tool shaft 102 and the angled portion 104 to facilitate sterilization of the handpiece 100. The tip portion 106 can be separable from the angled portion 104 to enable the tip portion 106 to be recoupled with the angled portion 104 at a different rotational angle. The tip portion 106 can be rotated with respect to the angled portion 104 without separating the tip portion 106 from the angled portion 104. The tip portion 106 can be rotated with respect to the angled portion 104 to provide the surgeon with improved access to the round window. For example, the surgeon, or a surgical robot, can adapt the default position of the tip portion 106 to account for variability between patient anatomies. The handpiece 100 can include gaskets or O-rings at the interface between the separable portions. The separable portions can be coupled together with snap-on connectors, friction-fit or press-fit connections, or Luer lock connections.

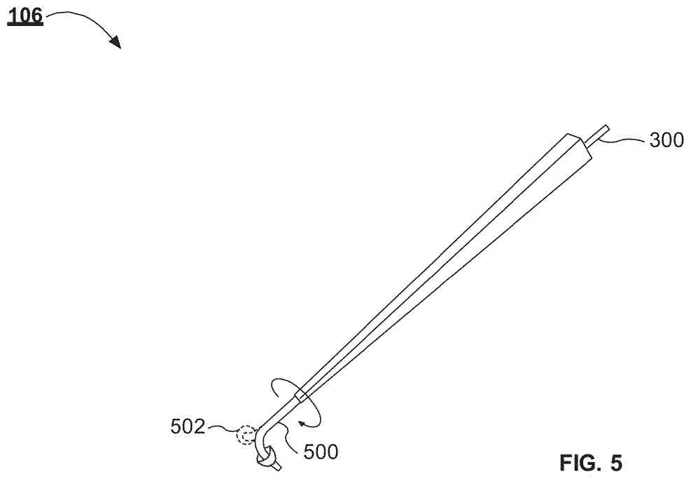

FIG. 5 illustrates an example tip portion 106 for the example handpiece 100. The tip portion 106 illustrated in FIG. 5 is separated from the angled portion 104 and the tool shaft 102 of the handpiece 100. The tip portion 106 can include a tip 500. The tip 500 can be, or can include, a portion of the microfluidic channel 300 extending from the body of the tip portion 106. In some implementations, all of the tip portion 106 can be rotated with respect to the angled portion 104. In other implementations, the tip 500 can be rotated within the tip portion 106. In either example, the tip 500 can be rotated from the position illustrated in FIG. 4 to a second position 502, illustrated by the dashed lines. As shown, the tip portion 500 can be bent or angled to facilitate seating the collar 108 with an anatomic structure in the middle ear of a patient, such as the round window. The bent portion of the tip 500 can form an angle between the outlet of the tip portion and 106 the body of the tip portion 106, where the angle is between about 90 degrees and about 175 degrees.

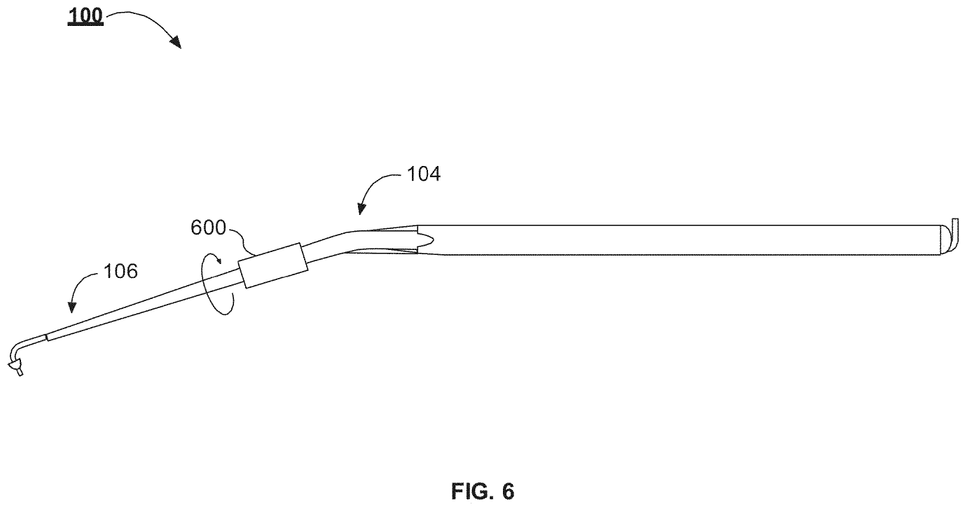

FIG. 6 illustrates an example handpiece 100 with a compression fitting 600. The compression fitting 600 can be knurled nut. The compression fitting 600 can couple the angled portion 104 with the tip portion 106. The compression fitting 600 can be loosened to enable the tip portion 106 to rotate with respect to the angled portion 104. Once the surgeon selects a degree of rotation, the surgeon can tighten the compression fitting 600 to lock the degree of rotation between the angled portion 104 and the tip portion 106 in place. In other implementations, the tip portion 106 and the angled portion 104 can be held together with a friction fit that enables the tip portion 106 to be rotated with respect to the tip portion 106. In such implementations, the tip portion 106 and the angled portion 104 can be rotated to a desired degree of rotation, and then pushed into a friction fit portion of the tool shaft 102 to fix the degree of rotation for a surgical procedure. The detachable tip and angled portion allows for the selection of tip materials and dimensions that conform to the anatomic properties of a patient undergoing a procedure using the handpiece 100.

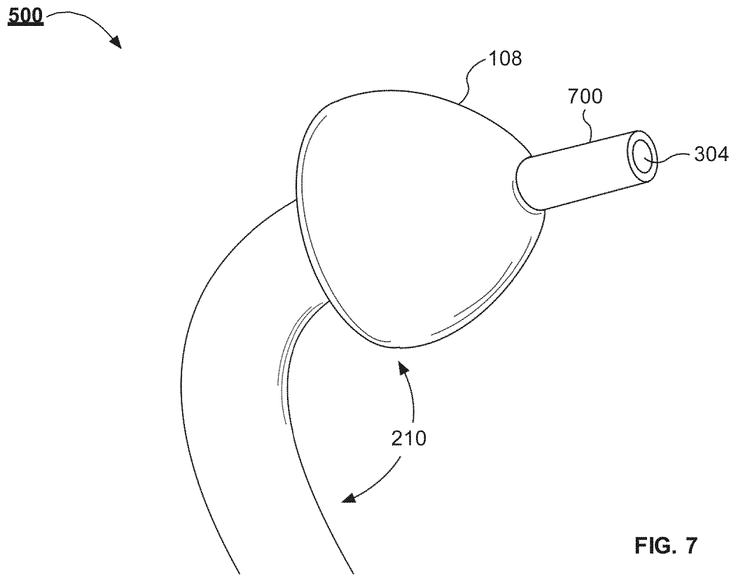

FIG. 7 illustrates an enlarged view of the tip 500 of the example handpiece 100. The tip 500 can include a needle end 700. The needle end 700 includes the outlet 304. The needle end 700 can be a blunt end or can be beveled to form a point. The needle end 700 can be configured to pierce the round window membrane or another anatomic structure in the ear of a patient. The needle end 700 can extend past the collar 108 by a length between about 1 mm and about 4 mm, between about 2 mm and about 3 mm, or between about 2.5 mm and about 3 mm. For example, the needle end 700 can have a length of 2.7 mm. The needle end 700 can have a gauge size between about 25 and about 30, between about 26 and about 30, or between about 27 and about 30. Once the collar 108 is seated into the round window only the needle end 700 projects into the cochlea. The collar 108 can control the depth the needle end 700 projects into the cochlea (e.g., the cochlea 112 depicted in FIG. 1, etc.).

The needle end 700 can prevent the needle end 700 from projecting too far into the cochlea. The needle end 700 can prevent the needle end 700 from projected too far into the cochlea and damaging the cochlea. The collar 108 can properly position the outlet 304 within the cochlea so that the therapeutic substance properly disperses through the cochlea (e.g., the cochlea 112 depicted in FIG. 1, etc.). For example, if the outlet 304 is positioned too shallow into the cochlea, the therapeutic substance can concentrate near the round window and not disperse through the cochlea. If the outlet 304 is position too deep into the cochlea, the needle end 700 can cause damage or trauma to the cochlea. In some implementations, the tip 500 is manufactured from a malleable material such that a surgeon can bend the tip 500 to alter the angle 210. The collar 108 can be coupled with the tip 500 with an adhesive. In some implementations, the tip 500 can include a groove in which the collar 108 is seated.

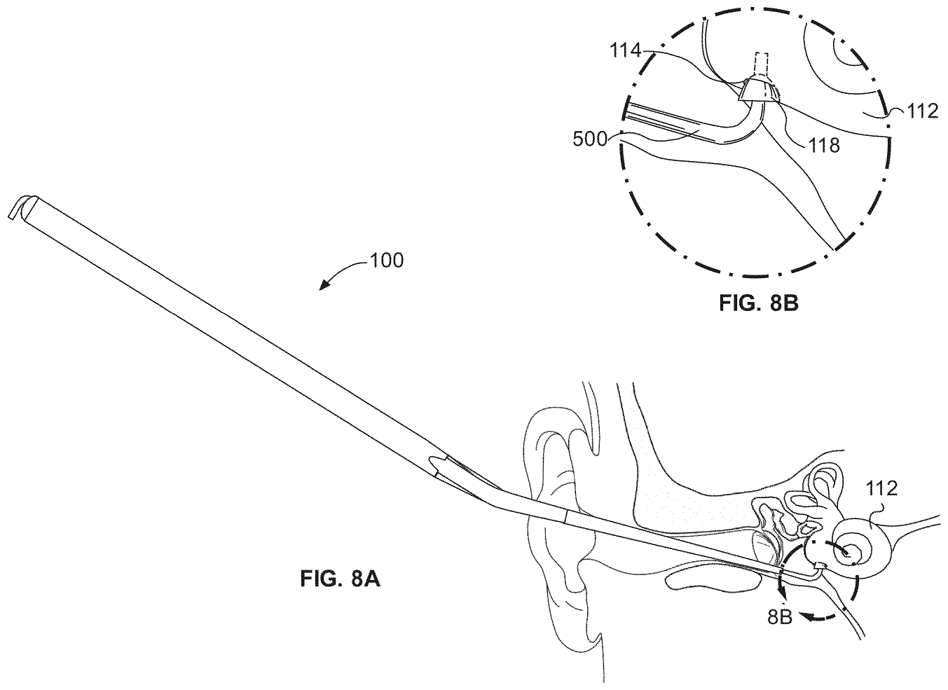

FIGS. 8A and 8B illustrate the tip 500 inserted into the round window. FIG. 8A illustrates the handpiece 100 inserted through the ear canal with the tip 500 inserted into the round window 114, or another type of anatomic structure in the ear of a patient. FIG. 8B illustrates an enlarged view, from FIG. 8A, of the tip 500 inserted into the round window 114. The tip 500 can be used to pierce the round window membrane. The tip 500 can be inserted into the round window 114. The collar 108 can be seated into the round window 114 and seal the round window 114 as fluid is injected into the cochlea 112. The collar 108 is tapered from a diameter smaller than the diameter of the round window 114 to a diameter that is wider than the diameter of the round window 114. When the collar 108 is depressed against the round window 114, the collar 108 can occlude the round window 114. The collar 108 can also be used to control the insertion depth of the tip 500 into the cochlea 112. For example, the collar 108 can prevent the tip 500 from being inserted into the cochlea past the collar 108. The portion of the collar 108 with a diameter wider than the diameter of the round window 114 can substantially stop the tip 500 from farther insertion of the tip 500 into the cochlea 112. Moving the collar 108 towards the outlet 116 of the tip 500 reduces the depth to which the tip 500 can be inserted. The collar 118 can prevent the tip 500 from being inserted too far into the cochlea 112.

FIG. 9 illustrates an example fluid reservoir 900 coupled with the example handpiece 100. The fluid reservoir 900 can be coupled with a pump that pumps the fluid stored in the fluid reservoir 900 through the microfluidic channel 300 of the handpiece 100 and out the outlet 304 of the handpiece 100. The fluid reservoir 900 can be coupled to displacement pump, syringe, syringe pump, or other type of mechanical, electric-mechanical, hydraulic, or pneumatic-driven actuator. The inlet 302 of the microfluidic channel 300 can coupled to the fluid reservoir 900 to enable the fluid to be introduced to the microfluidic channel 300. In some implementations, the fluid reservoir 900 can be separable from the handpiece 100. The fluid reservoir 900 can include a septum that the inlet 302 pierces when a user attaches the fluid reservoir 900 to the handpiece 100. In other implementations, the fluid reservoir 900 can be a component of the handpiece 100 that is filled with a fluid prior to use. The fluid reservoir 900 can include a septum through which the fluid reservoir 900 is loaded. For example, a syringe can be loaded with the therapeutic substance. The needle of the syringe can be inserted through the septum and the therapeutic substance injected into the fluid reservoir 900.

FIG. 10 illustrates an example fluid reservoir 900 coupled with a handpiece 100. The fluid reservoir 900 can include a self-contained pumping system. The self-contained pumping system can pump a fluid from the fluid reservoir to the outlet 304. The fluid reservoir 900 of the self-contained pumping system can be refill from an external reservoir, for example via a detachable connection. The detachable connection can include a closeable port, such as a threaded port or a valve, with a connector for an external hose or channel. The external hose or channel can connect to an external source of fluid, which can travel through the external hose when connected to fluid reservoir 900 to fill the fluid reservoir 900. The fluid reservoir 900 can be configured to store a predetermined volume of fluid, such as a drug compound. Thus, the device 100 depicted in FIG. 10 can be used to provide precise volumes or doses of compound by providing only what is stored in the fluid reservoir 900 of the self-contained pumping mechanism.

It should be understood that the handpiece 100 can include additional or different features than those depicted in FIGS. 9 and 10. For example, the handpiece 100 may include a guiding light positioned near the tip to allow a physician to more easily see the anatomy of an ear of the patient while using the handpiece 100. The handpiece 100 can also be integrated with a micropump or a fluid reservoir in other manners than those depicted in FIGS. 9 and 10. For example, a micropump can be partially inserted into the handpiece 100. In some other implementations, a micropump can be attached to a portion of the handpiece 100, for example using a press fit, friction fit, mechanical fasteners, or any other suitable means of attachment. In some implementations, the handpiece 100 can include a pump compartment configured to receive at least a portion of a micropump and/or a fluid reservoir.

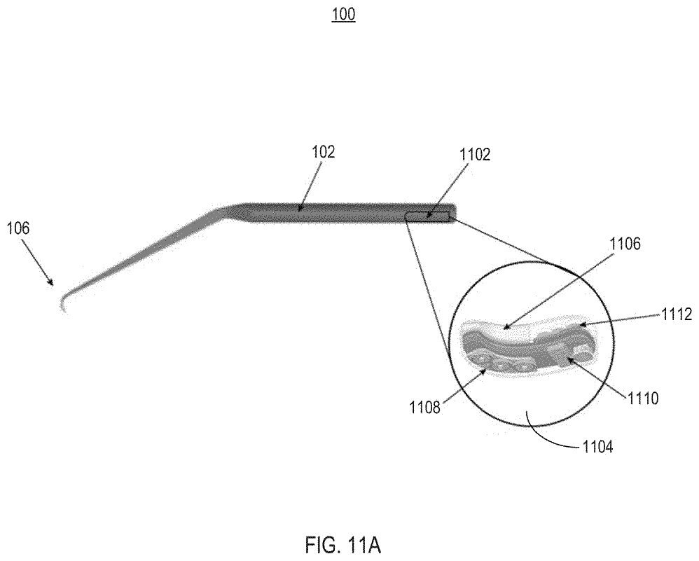

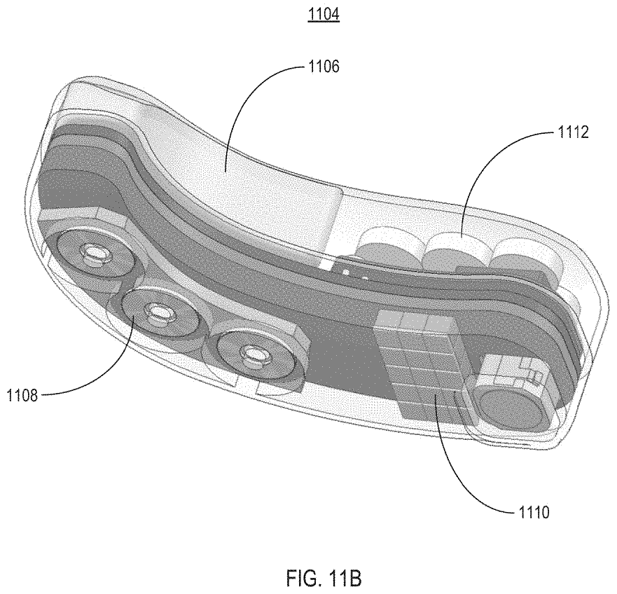

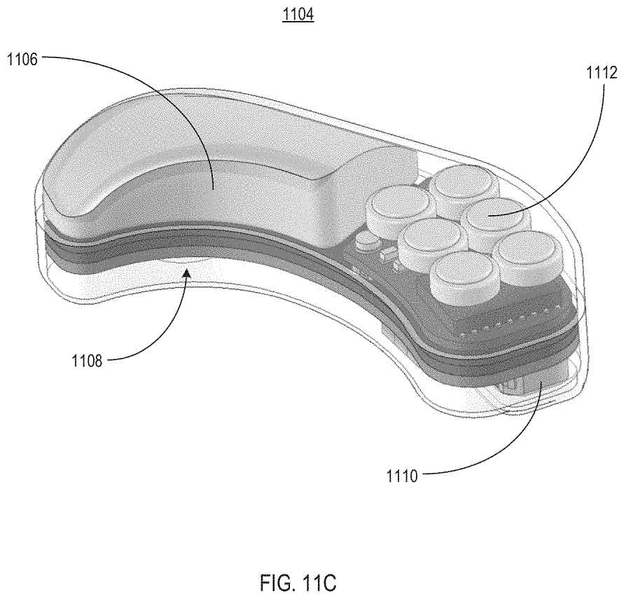

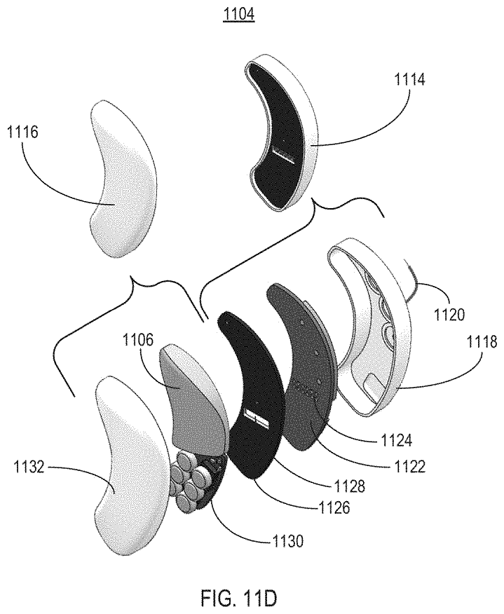

FIG. 11A illustrates an example handpiece 100 having an integrated micropump 1104. FIGS. 11B-11D illustrate various views of the micropump 1104. Referring now to FIG. 11A, the handpiece 100 can be similar to the handpiece shown and described above in connection with FIG. 1, among others, and like reference numerals refer to like elements in the drawings. The handpiece 100 includes a tool shaft 102 and a tip portion 106 extending form an angled portion coupled with the tool shaft 102. The handpiece 100 also includes pump compartment 1102. The pump compartment 1102 can be configured to store, house, or receive a micropump 1104, which may also be referred to herein as a pump 1104.

The pump compartment 1102 can be or can include a void or recess formed within an end portion of the tool shaft 102 of the handpiece 100. The void or recess can be shaped in a manner that provides sufficient space to house the micropump 1104. In some implementations, the handpiece 100 can also include a cover to seal an opening of the pump compartment 1102 after the micropump 1104 has been installed. In some implementations, the micropump 1104 can be installed permanently within the pump compartment 1102 of the handpiece 100. For example, the micropump 1104 can be arranged in a fixed manner and may not be intended to be removed from the pump compartment 1102 of the handpiece 100. In some other implementations, the micropump 1104 can be installed in a removable fashion. For example, the micropump 1104 may snap into place within the pump compartment 1102 and may be configured to be removable from the pump compartment 1102. The micropump 1104 may also be configured to be secured within the pump compartment 1102 via mechanical fasteners, adhesive, or other means of attachment as described herein.

Referring briefly now to FIGS. 11B and 11C, the micropump 1104 can include a fluid reservoir 1106. The fluid reservoir 1106 can be configured to store a fluid, such as a liquid sample containing a therapeutic substance. In some implementations, the fluid reservoir 1106 can be accessible via a port or inlet in the handpiece 100 to allow a fluid sample to be introduced into the fluid reservoir 1106. The fluid reservoir 1106 can be re-sealable to prevent the fluid sample from spilling out of the fluid reservoir. Similar to the fluid reservoir 900 described above in connection with FIG. 9, the fluid reservoir 1106 can be used to store fluid to be introduced into an ear of the patient via the handpiece 100. For example, the micropump 1104 can be configured to pump the fluid stored in the fluid reservoir 1106 along a length of the handpiece 100 towards the tip portion 106 and out of the outlet 304. To accomplish this, the micropump 1104 can include electromagnetic actuators 1108, electronic components 1110, and batteries 1112.

The electromagnetic actuators 1108 can be actuators that are configured to move or rotate in response to electric signals, such as those received from the electronic components. The electromagnetic actuators 1108 can include an inductor magnetically coupled to a magnetic substance. The magnetic substance can be configured to move, or actuate, in response to a changing magnetic field in the inductor. The inductor of the electromagnetic actuator can be a copper coil, or another type of embedded induction device. The electromagnetic actuators 1108 can be configured to cause valves between channels in the micropump 1104 to open or close. The electromagnetic actuators 1108 can cause pumps, such as microchannel peristaltic pumps within the micropump 1104, to actuator or turn. Thus, the electromagnetic actuators can cause fluid to flow into, through, and out of the micropump 1108 in a controlled manner, as governed by the electronic signals that induce a magnetic field in the inductors of the electromagnetic actuators 1108. The signals that cause the electromagnetic actuators 1108 to actuate can be received from the electronic components 1110.

For example, the electronic components 1110 can include electronic switches or transistors, such as metal-oxide silicon field-effect transistors (MOSFETS), bipolar junction transistors, or other types of electronically actuated switches. The transistors or switches of the electronic components 1110 can route power from the batteries 1112 to the electromagnetic actuators 1108 to induce a magnetic field in the electromagnetic actuators 1108, thus causing the electromagnetic actuators 1108 to actuate according to their configuration (e.g., open or close a valve, cause a pump to move fluid, etc.). The electronic components 1110 can be powered, for example, by energy received in a circuit formed with the batteries 1112. The electronic components 1110 can include a control circuit that actuates the electromagnetic actuators in response to one or more events, such as a predetermined sequence, a button input (e.g., via a button on the exterior of the handpiece 100, etc.), or other type of control circuit.

The battery 1112 can be any type of battery, such as a lithium-ion battery, a metal-nickel-hydride battery, or an alkaline battery, among others. The battery 1112 can be one or more batteries, which can form an electric circuit with one or more of the electronic components 1110 or the electromagnetic actuators 1108. The battery 1112 can be configured to be rechargeable via a charging mechanism, such as an inductive charging mechanism or an external charging port. The battery 1112 may also be disposable, such that when the battery 1112 loses its charge, the micropump 1104 may be disposed of and replaced with a micropump 1104 having a fully charged battery.

As described above, a fluid reservoir can be coupled with a standard syringe pump, a peristaltic pump, or other pump that may be interfaced with the fluid reservoir via a network of fluidic tubing. However, such arrangements can add additional dead volume to the system, resulting in wasted therapeutic substance, delays in the time at which therapeutic substance reaches the patient cochlea, potential difficulties in placement and management of bulky pump and tubing assemblies, and increased cost. In some cases, the therapeutic substance can be very expensive. Thus, increased dead volume within the system can lead to large increases in cost of treatment.

Referring back now to FIG. 11A, the handpiece 100 solves such technical issues by bringing the micropump 1104 closer to the interface to the inner ear by fully integrating with the handpiece 100 inside the pump compartment 1102. This significantly reduces dead volume in the system while maintaining precise control over dosing. The reduction in dead volume can greatly reduce the cost of therapeutic substance used for a given treatment, and the configuration of the handpiece 100 shown in FIG. 11A can simplify the surgical procedure and eliminate modes for errors or operator mistakes, relative to configurations that do not include an integrated micropump 1104 inside the pump compartment 1102 of the handpiece 100. The handpiece 100 having the integrated micropump 1104 can also be simpler to implement clinically and easier to work with than a system that requires an external connection to a pump. In some implementations, the handpiece 100 of FIG. 11A can be similar to the handpiece depicted in FIGS. 9 and 10.

In some implementations, the design of the handpiece 100 can be altered to avoid blocking the ability to visualize the middle and inner-ear structures during the surgery. In some implementations, the handpiece 100 can further include integration of flow sensing capabilities into the handpiece 100. For example, one or more sensors (e.g., flow sensors) that may be incorporated into the micropump 1104, separately incorporated within the pump compartment 1102 of the handpiece 100, or incorporated elsewhere in the handpiece 100. In some implementations, the handpiece 100 or the micropump 1104 may include multiple fluid reservoirs. For example, the handpiece 100 can include one or more fluid mixing chambers, which may be configured to mix any combination of one or more fluids or one or more powders to produce a therapeutic substance to be delivered via the micropump 1104. For example, such an arrangement could extended a shelf life of a drug and could ease distribution of the systems.

In some implementations, the handpiece 100 can also be configured to receive a blister pack containing a fluid to be delivered to a patient. The blister pack can be received into the pump compartment 1102 at the time of the procedure. The blister pack may serve as its own fluid reservoir. Thus, the micropump 1104 may not have an integrated fluid reservoir such as the fluid reservoir 1106, but instead can interface with a blister pack and can pump fluid contained in the blister pack. A blister pack can include a predetermined volume or dosage of a drug compound or another type of fluid that can be delivered using the handpiece 100. Using a blister pack can allow a physician to provide only a known, pre-measured dosage of a drug for a particular procedure, rather than requiring manual drug compound or fluid measurement. The micropump 1104 may interface with the blister pack by piercing a portion of the blister pack containing a desired fluid. When piercing the blister pack, the micropump 1104 can create a fluid-tight seal with the blister pack while creating a fluid connection between the blister pack and the other components of the micropump 1104.

In some implementations, the micropump 1104 can be integrated in with the handpiece 100 in a manner different than that depicted in FIG. 11A. For example, the micropump 1104 need not be entirely contained within the pump compartment 1102. In some implementations, the micropump 1104 can be integrated with the handpiece 100 such that only a portion of the micropump 1104 is positioned within the handpiece 100 (e.g., inside the pump compartment 1102), while a remaining portion of the micropump 1104 may be positioned outside of the handpiece 100. For example, the micropump 1104 may appear similar to the fluid reservoir 900 depicted in FIGS. 9 and 10, in that portions of the micropump 1104 can be external to the handpiece 100 rather than integrated into the tool shaft 102 of the handpiece 100. In still other implementations, the micropump 1104 can be integrated with the handpiece 100 such that substantially all of the micropump 1104 is positioned outside of the handpiece 100. For example, the handpiece 100 and the micropump 1104 can be designed such that the micropump 1104 can be attached to an exterior surface of the handpiece 100.

Referring now to FIG. 11D, a detailed view of an implementation of the micropump 1104 is illustrated. The implementation of the micropump 1104 depicted in FIG. 4D can be used in connection with the handpiece 100, for example as illustrated in the arrangement of FIG. 11A. However, in some other implementations the micropump 1104 can be used on its own without the handpiece 100. For example, the micropump 1104 can be configured to be implanted or attached to a patient for treatment, and the micropump 1104 can pump a fluid containing a therapeutic substance into an ear of the patient without the use of the handpiece 100.

The micropump 1104 can include an implanted module 1114 and a drug module 1116. The implanted module 1114 and the drug module 1116 can be individual components that are separable from one another. The implanted module 1114 can include a case 1118. In some implementations, in use with a patient, the case 1118 of the implanted module 1114 can face towards the head of the patient. The implanted module 1114 can be attached to or implanted or embedded within the head or ear of the patient. In some implementations, the implanted module 1114 can be wearable by the patient for an extended period of time. For example, the implanted module 1114 can be configured to remain worn, attached, or implanted within the patient for a period of days, weeks, months, or longer. When the implanted module 1114 is worn or implanted in the patient, the cannula 1120 can protrude into an ear of the patient.

The implanted module 1114 also includes a board 1122. The board 1122 can include pump electronics, which can include the electronic components 1110, and actuators (e.g., the electromagnetic actuators 1108, etc.) for controlling pumping of a fluid out through the cannula 1120. For example, the electromagnetic actuators 1108 and the electronic components 1110 can be mounted to the board 1122. The board 1122 can include an interface 1124. The interface 1124 can include electrical interface elements and fluidic interface elements. The interface 1124 can be configured to couple with other portions of the 1104 to receive electrical signals and fluid to be pumped through the cannula 1120. The implanted module 1114 can also include a board 1126. The board 1126 can serve as mounting surface for pump fluidic components, such as channels and valves. The board 1126 can also include an opening 1128. The opening 1128 can be aligned with the interface 1124 of the board 1122. Thus, the interface 1124 can access other components on an opposite side of the board 1126, such as components of the drug module 1116, via the opening 1128.

The micropump 1104 can also include a drug module 1116. The drug module 1116 can include a board 1130. The board 1130 can serve as a mounting surface for batteries, such as the batteries 1112, as well as other control electronics for the micropump 1104. The board 1130 can also include the fluid reservoir 1106, which can store a fluid sample (e.g., a sample of fluid containing a drug or other therapeutic substance), as described above. An outer case 1132 of the drug module 1116 shields the board 1130 and its components from the outside environment. The implanted module 1114 and the drug module 1116 are shown in an exploded view in FIG. 11D. Thus, when sealed (e.g., when the case 1118 and the case 1132 are brought together), the interior components can be enclosed within a housing formed by the case 1118 and the case 1132.

The micropump 1104 can be used in either an acute delivery scenario or a chronic delivery scenario. For example, in an acute delivery scenario, the implanted module 1114 can be implanted in or mounted on the patient, with the cannula 1120 protruding into the ear of the patient. The implanted module 1114 can be implanted or mounted in a manner intended for long-term wear. When the patient visits an office of a physician for treatment, the physician can attach the drug module 1116 to the implanted module 1114. The fluid reservoir 1106 of the drug module 1116 can be filled with a fluid to be used for treating the patient. The drug can be administered to the patient for a relatively short period of time (e.g., seconds, minutes, or hours). In some implementations, the drug may be administered only during a time period that coincides with the patient's visit to the physician's office. Then, the physician can remove the drug module. The implanted module 1114 can remain implanted or mounted to the patient. In some implementations, an exposed portion of the implanted module 1114 can be covered until the next acute delivery. Thus, in the acute delivery scenario, the patient may only wear the implanted module 1114 long-term, while the drug module 1116 is attached for shorter periods of time only during scheduled acute deliveries.

In a chronic delivery scenario, the implanted module 1114 can be implanted or mounted to the patient, similar to the acute delivery scenario described above. A physician can also attach the drug module 1116 to the implanted module 1114. However, unlike the acute delivery scenario, the drug module 1116 can remain attached to the implanted module 1114 for a long period of time (e.g., days, weeks, months, or longer) in the chronic delivery scenario. Thus, the patient can wear both the implanted module 1114 and the drug module 1116 long-term. The drug can be administered in accordance with a predetermined dosage schedule over time, and not only while the patient is visiting the physician's office. When the dosage schedule has ended or when the fluid reservoir 1106 becomes depleted, the patient can again visit the physician, who can either refill the fluid reservoir 1106 or replace the entire drug module 1116 with a new drug module 1116 in order for additional dosages to be administered to the patient.

Thus, the components of the micropump 1104 can be arranged in a layered form factor in which electronic and fluidic components are mounted to boards (e.g., the board 1122, the board 1126, and the board 1128) that form a stack which can be enclosed between the inner case 1118 and the outer case 1132. This form factor can appropriate for human clinical use, and can allow a patient to wear at least a portion of the device (e.g., the implanted module 1114 or the drug module 1116, or both) more comfortably and for longer periods of time, as compared with other form factors. It should also be understood that the micropump 1104 can be used with the handpiece 100 as shown in FIG. 11A in some implementations. Thus, the implanted module 1114 and the drug module 1116 can be used with the hand piece 100 and may not be implanted or worn directly by the patient.

The separation of the implanted module 1114 from the drug module 1116 can have other technical benefits as well, whether the micropump 1104 is used with the handpiece 100 or worn by the patient. For example, the drug module can include the fluid reservoir 1106 containing the drug to be administered as well as control electronics, which can be used to store a drug delivery program, logic, or other executable code to serve as instructions for the micropump 1104 for administering the drug to the patient. In such an arrangement, the drug delivery program and the drug itself are physically present in the same module (e.g., the drug module 1116) and on the same board (e.g., the board 1130). As a result, the drug cannot be separated from the drug delivery program, which can increase patient safety by reducing the likelihood of an inadvertent mismatch between the drug and the drug delivery program. The implanted module 1114 can be mounted semi-permanently to the patient, and can be coupled with the drug module 1116 using a sterile connection. In some implementations, the micropump 1104 can be operated in an infusion-only mode. In some other implementations, the micropump 1104 can be operated in a reciprocating mode, which can enable zero-net-volume delivery.

In some implementations, the computer code or logic implemented by the control electronics of the board 1130 can be programmable, selectable, or otherwise configurable by a user, such as a physician. For example, the physician may program the control electronics to achieve a desired or predetermined drug delivery schedule. The drug delivery schedule may include any number of selectable or configurable parameters, such as flow rates, drug administration times, or operational modes (e.g., infusion only, reciprocating, etc.). Thus, the micropump 1304 can be fully programmable in a manner that allows a physician to determine an appropriate drug delivery schedule, develop a drug delivery program in accordance with the drug delivery schedule, and store the drug delivery program within control electronics of the board 1130 to enable the micropump 1104 to administer the drug to the patient according to the drug delivery schedule in an autonomous fashion. The programmable circuitry can include a programmable timer, which can be a timer integrated with a microcontroller or embedded central processing unit. The programmable timer can cause one or more circuits (e.g., the electronic circuits 1110, any other circuitry described herein, etc.) to generate one or more electronic signals that cause the pumps or valves of the micropump 1104 to move fluid through the system.

FIG. 12 illustrates a schematic view of an example micropump 1200. In some implementations, the micropump 1200 can be an implementation of the at least a portion of the micropump 1104 shown in FIGS. 9, 10, and 11A-11D. For example, the micropump 1200 can be used along with the handpiece 100 (e.g., in an arrangement similar to that shown in FIG. 11A, in which the micropump 1200 is integrated with the handpiece 100). In some implementations, at least a portion of the micropump 1200 can be worn by a patient, with or without the use of the handpiece 100. The micropump 1200 can include the drug reservoir 1201 and a fluid storage capacitor 1202. A drug-containing fluid can be dispensed from the micropump 1200 via the outlet 304. The micropump 1200 can include a pump 1206. The micropump 1200 can include a plurality of valves 1208 and fluid capacitors 1204.

The micropump 1104 can be a multilayered device. The micropump 1200 can include fluid routing layers. For example, the fluid routing layers can include the drug reservoir 1201, fluid storage capacitor 1202, fluid capacitors 1204, the channels 1210, and a loading chamber 1212. The micropump 1200 can include one or more active layers. The active layers can include the actuators of the valves 1208 and the pump 1206, the controller that controls the valves 1208 and the pump 1206, and a power source for powering the micropump 1200. The fluid routing layers can be separated from the active layers by a membrane. The fluid routing layers can include polyetherimide (PEI). The membrane separating the fluid routing layer and the active layers can include a flexible membrane, such as polyimide and Viton.

The micropump 1200 can include the drug reservoir 1201. The drug reservoir 1201 can be similar to the fluid reservoir 1106 of FIGS. 11A-11D. In some implementations, the drug reservoir 1201 can be machined (e.g., laser etched) into one or more of the fluid routing layers. The drug reservoir 1201 can be configured as a serpentine or other channel structure. The drug reservoir 1201 can be configured as a channel with an inlet and an outlet such that a fluid can be pumped into the inlet to force the drug from the outlet of the drug reservoir 1201 and into one of the channels 1210. The drug reservoir 1201 can have a channel width between about 300 μm and about 1200 μm, between about 400 μm and about 1000 μm, between about 500 μm and about 900 μm, between about 600 μm and about 800 μm, or between about 700 μm and about 800 μm. The drug reservoir 1201 can have a channel height between about 300 μm and about 1200 μm, between about 400 μm and about 1000 μm, between about 500 μm and about 900 μm, between about 600 μm and about 800 μm, or between about 700 μm and about 800 μm. The drug reservoir 1201 can have a total channel length between about 300 mm and about 100 mm, between about 300 mm and about 800 mm, or between about 300 mm and about 600 mm.

The micropump 1200 can include a fluid storage capacitor 1202. The fluid storage capacitor 1202 can be a cylinder formed in the fluid routing layer. The fluid storage capacitor 1202 can have a diameter of between about 10 mm and about 20 mm, between about 12 and about 18 mm, or between about 14 and about 16 mm. The fluid storage capacitor 1202 can be configured to store fluid withdrawn from the inner ear of the patient. The fluid storage capacitor 1202 can also provide fluid to the inlet of the drug reservoir 1201 to force the drug out of the outlet of the drug reservoir 1201.

The micropump 1200 can also include a plurality of fluid capacitors 1204. The fluid capacitors 1204 can be machined in line with the fluid channels 1210 and loading chamber 1212 of the fluid routing layer. The fluid capacitors 1204 can have a diameter of between about 2 mm and about 10 mm, between about 2 mm and about 8 mm, between about 2 mm and about 6 mm, or between about 4 mm and about 6 mm. The fluid storage capacitor 1202 and the fluid capacitors 1204 can have a ceiling formed by the membrane separating the fluid routing layers and the active layers.

The fluid capacitors 1204 can improve power efficiency, help to regulate peak flow rates, and provide fluid storage. For example, the channels 1210 of the micropump 1200 can have relatively high fluid resistances, which can cause a relatively large time constant associated with expelling fluid from the micropump 1200. Accordingly, with a relatively large time constant, the valves 1208 may need to be powered for several seconds to open the valves and to enable the pump chamber to have time to fully drain or fill. The fluid capacitors 1204 that are in line with the fluid channels 1210 have lower fluid resistance and can enable relatively fast transfer of fluid into and out of the pump chamber followed by passive fluid flow associated with the pressure equilibration of the fluid capacitors 1204. This can reduce the amount of time valves 1208 are held open (to on the order of tens of milliseconds) and can reduce power consumption. The fluid capacitors 1204, for example the fluid capacitor 1204 near the outlet 304, can attenuate flow rate bursts generated by pump strokes and reduce large peak flow rates.

The micropump 1200 can include one or more pumps 1206. The pump 1206 can include an actuator in the active layers of the micropump 1200. The actuator can hold electromagnets in place. When the electromagnets are unpowered, springs can keep the actuator heads pressed against the polyimide membrane. Pressure against the polyimide membrane presses the Viton layer against an opening to the cylinder of the valve 1208 formed in the fluid layer and forms a fluidic seal that closes the valve of the pump 1206.

Cycling the actuator of the pump 1206 can result in fluid displacement in the fluid chamber of the pump 1206. The valves 1208 can be cycled (e.g., opened or closed) to control the direction of the fluid flow through the micropump 1200. For example, for each stroke type, one valve can act as an intake valve and another valve can act as an expulsion valve. At the beginning of a pump stroke, the intake valve opens, and then the pump actuator is powered resulting in fluid being drawn into the pump chamber from an adjacent fluidic capacitor. Next, the intake valve closes. Then the expulsion valve opens, followed by deactivation of the pump actuator, resulting in fluid being pushed out of the pump chamber into a different fluidic capacitor. Finally, the expulsion valve closes. Depending on which valves are chosen as the intake and expulsion valves, the pump can produce three different types of pump strokes: infusion (e.g., fluid is pumped out of the micropump 1200), withdrawal (e.g., fluid is pumped from an external source into the micropump 1200), and drug refresh or priming (e.g., fluid is pumped into the loading chamber 1212 to be pumped out of the micropump 1200 at the end infusion stroke).

The micropump 1200 can include one or more valves 1208. The valves 1208 can have a construction similar to the pump 1206. For example, the valves 1208 can include a cylinder chamber formed into the fluidic layers. The valves 1208 can include an actuator in the active layers that holds electromagnets in place. When the electromagnets are unpowered, the valves can be held in a closed position by a spring that forces the actuator against the membrane to form a seal in the opening of the cylinder chamber of the valve 1208. Activation of the actuator can force the electromagnets against the spring and away from the membrane to enable fluid to flow through the valve 1208.

In some implementations, the micropump 1200 can instead be driven by stored air or liquid pressure, mechanical strain, temperature-varying mechanical properties, or other modalities that can obviate the need for precision electromechanical devices, such as the electromagnetic actuators 1108.

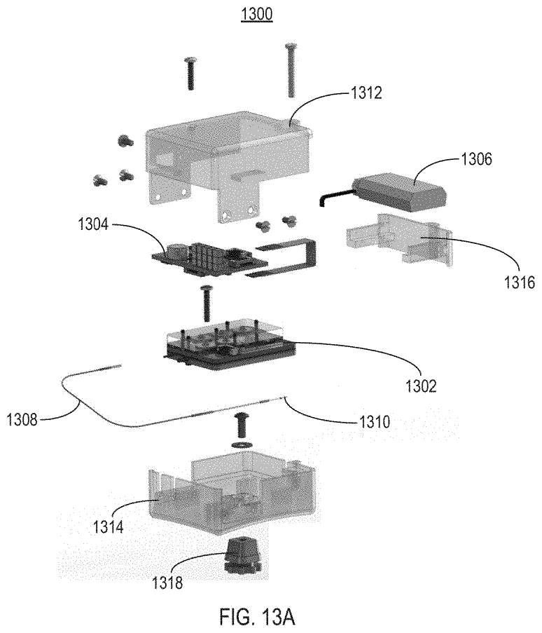

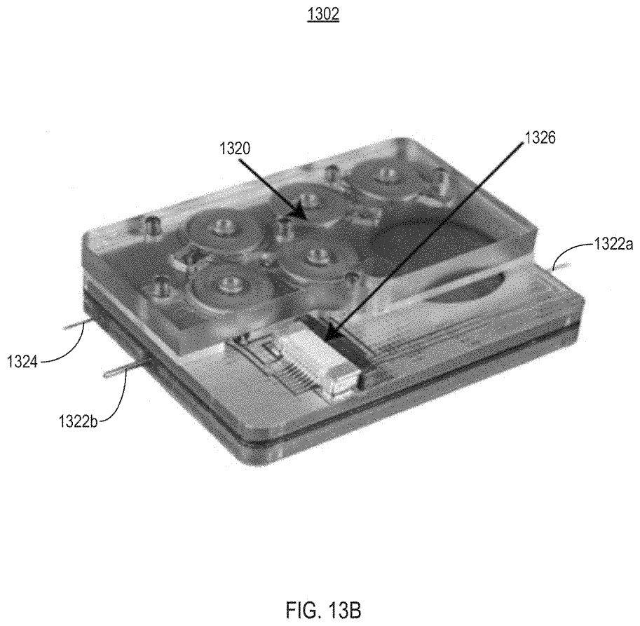

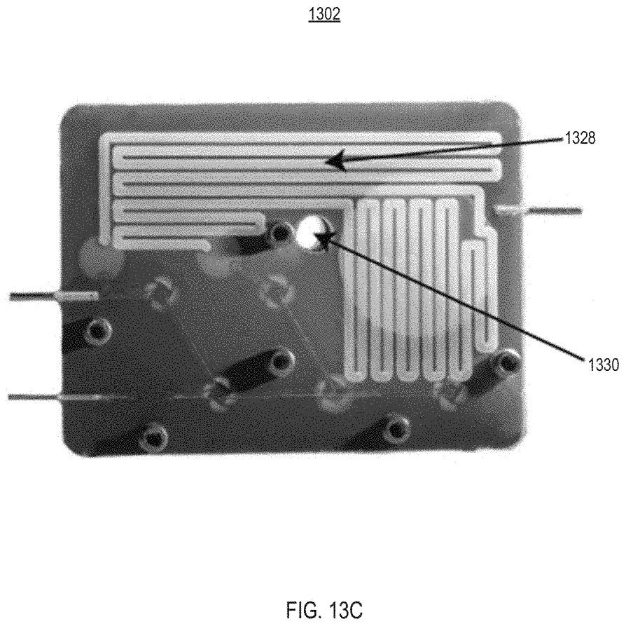

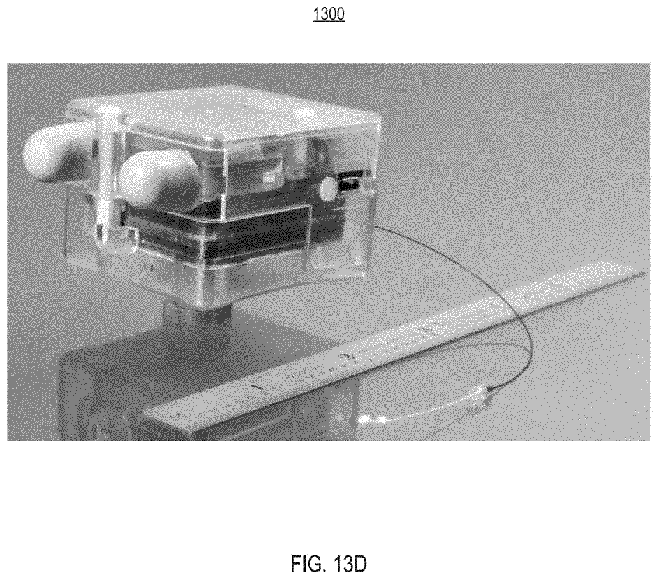

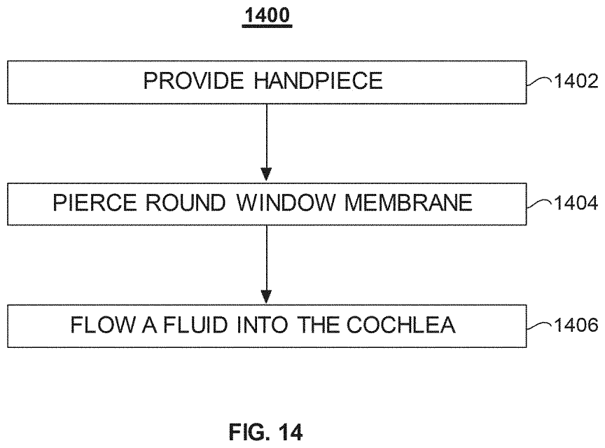

FIGS. 13A, 13B, 13C, and 13D depict various views of components of an example wearable device 1300 for administering a drug. Referring now to FIG. 13A, depicted is an exploded view of the device 1300. The device 1300 can include a micropump 1302. In some implementations, the micropump 1302 can be similar to the pump 1200 described above in connection with FIG. 12 or the micropump 1104 described above in connection with FIGS. 11A-11D. The device 1300 can also include a controller board 1304. The controller board 1304 can be communicatively coupled with the micropump 1302. The controller board 1304 can be powered by a battery 1306. The controller board 1304 can control the operation of the micropump 1302. For example, the controller board 1304 can store computer code or logic to implement a set of instructions for controlling the micropump 1302. In some implementations, the computer code or logic can be programmable, selectable, or otherwise configurable by a user, such as a physician. For example, the physician may program the controller board 1304 to select parameters such as times at which the micropump 1302 should be activated to administer a drug to the patient, flow rates at which the micropump 1302 should administer the drug, etc. Tubing 1308 can be fluidly coupled with the micropump 1302 and can transport fluid from the micropump 1302 to the cannula 1310 at an opposite end of the tubing 1308. In some implementations, the tubing 1308 can be formed from a polymer material, such as polyether ether ketone (PEEK).

FIG. 13B shows a top perspective view of the micropump 1302, and FIG. 13C shows a bottom perspective view of the micropump 1302 of the device 1300. As shown, the micropump 1302 can include electromagnetic actuators 1320, which can be similar to the electromagnetic actuators 1108 shown in FIGS. 11A-11D. The micropump 1302 includes two priming inlets 1322a and 1322b, as well as an outlet 1324. The micropump also includes an integrated drug reservoir 1328, which can be similar to the fluid reservoir 1106 or the drug reservoir 1201. The outlet 1324 of the micropump 1302 can be fluidly coupled with a cannula, for example using tubing such as the tubing 1308 shown in FIG. 13A. The micropump 1302 can include an electrical connection 1326, which can be communicatively coupled with the controller board 1304 to allow electrical signals from the controller board 1304 to control operation of the micropump 1302. Thus, in some implementations, the micropump 1302 can include features similar to those included on the boards 1124 and 1128 of FIG. 11D, the controller board 1304 can include features similar to those included on the board 1130 of FIG. 13D, and the micropump 1302 can communicate with the controller board 1304 using an interface similar to the interface 1124 of FIG. 11D.

Referring back now to FIG. 13A, the components of the device 1300 can be enclosed within a housing, which may also be referred to as a pod. The housing can include a lid 1312 and a base 1314. In some implementations, the lid 1312 and the base 1314 can be configured to interlock or otherwise couple with one another to partially enclose the micropump 1302 and the controller board 1304. The housing can also include a battery door 1316 which can be opened to provide access to the battery 1306. The housing can also be configured to couple with a pedestal 1318. FIG. 13D depicts a perspective view of the device 1300 assembled with the housing enclosing the other components described herein above in conjunction with FIGS. 13A, 13B, and 13C.

FIG. 14 illustrates a block diagram of an example method 1400 to flow a fluid into the cochlea. The method 1400 can include providing a handpiece (ACT 1402). The method 1400 can include piercing a round window membrane (ACT 1404). The method 1400 can include flowing a fluid into the cochlea (ACT 1406).

As set forth above, the method 1400 can include providing a handpiece (ACT 1402). The handpiece 100 can be any handpiece described herein. For example, the handpiece 100 can include a tool shaft that includes a first distal end, a first proximal end, a first fluidic channel, and a first longitudinal axis. The handpiece 100 can include an angled portion that can include a second distal end, a second proximal end coupled with the first distal end, a second fluidic channel in communication with the first fluidic channel, and a second longitudinal axis defining an obtuse angle with the first longitudinal axis. The handpiece 100 can include a tip portion projecting from the angled portion and comprising an outlet and a third fluidic channel in communication with the second fluidic channel. The handpiece 100 can include a collar coupled with the tip portion. The handpiece 100 can be integrated with a micropump and a fluid reservoir, as described herein in conjunction with FIGS. 11A-11D. For example, the handpiece 100 can include a pump compartment configured to receive a micropump and fluid reservoir, as shown in FIG. 11A. The handpiece 100 can also be integrated with the micropump in other ways. For example, a micropump can be configured to snap onto a portion of the handpiece 100.

The method 1400 can include piercing a round window membrane (ACT 1404). The round window membrane can be pierced with the tip portion of the handpiece 100. For example, the provided handpiece 100 can be inserted through the ear canal. The angled portion 104 of the handpiece 100 can be configured to enable transcanal access of the round window. The tip 500 of the tip portion 106 can be angled to position the needle end 700 substantially perpendicular to the round window and round window membrane. The needle end 700 can be pressed against the round window membrane to pierce the round window membrane. The collar 108 can prevent the needle end 700 from projecting too far into the cochlea and causing damage to the cochlea.

The collar 108 can seat into the round window to seal the round window as the fluid is injected into the cochlea. Based on the anatomy of the patient, a surgeon can set a rotational offset between the tip portion and the angled portion of the handpiece 100 to enable the needle end 700 to access the round window. Also based on the anatomy of the patient, the surgeon can set the angle 210 between the needle end 700 and the tip portion such that the outlet 304 is positioned substantially perpendicular to the round window and round window membrane. CT or Mill scans of the middle and inner ear of the patient can be conducted. The surgeon can measure the anatomical angles of the inner and middle ear of the patient to select the angle 210 of the tip portion 106. Also, based on the CT or MRI scans the surgeon can select the length of the needle end 700 such that when the collar 108 is seated into the round window the outlet 304 is properly positioned within the cochlea. The proper position of the outlet 304 can be a depth into the cochlea that does not cause damage to the cochlea but enables distribution of the fluid through the cochlea.

The method 1400 can include flowing a fluid into the cochlea (ACT 1406). A pump can pump the fluid from a fluid reservoir 900, through the microfluidic channel 300, and into the cochlea via outlet 304. In some implementations, the pump can be external to the handpiece 100. In other implementations, fluid reservoir 900 can include a self-contained pump that pumps the fluid from the fluid reservoir 900 to the outlet 304. The method 1400 can include drilling, or otherwise forming, a ventilation hole in the stapes footplate. The ventilation hole can enable the release of pressure from the cochlea as the pump flows the fluid into the cochlea. Drilling the ventilation hole can be performed using a surgical tool such as a drill or other type of boring tool.

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the terms “about” and “substantially” will be understood by persons of ordinary skill in the art and will vary to some extent depending upon the context in which they are used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation or embodiment, and references to “an implementation,” “some implementations,” “one implementation,” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description, or any claim are followed by reference signs, the reference signs have been included to increase the intelligibility of the drawings, detailed description, and claims. Accordingly, neither the reference signs nor their absence has any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. A handpiece device to deliver a fluid into an inner ear, comprising:
a shaft comprising a first fluidic channel;
a tip portion coupled with a first end of the shaft and comprising an outlet and a second fluidic channel in fluid communication with the first fluidic channel; and
a collar coupled with the tip portion a predetermined distance from the outlet, the collar configured to seat with an anatomic structure and control a distance that the tip portion can project into a cochlea when the tip portion is inserted into an ear canal,
wherein the shaft is integrated with a micropump and a fluid reservoir, the micropump configured to pump a fluid from the fluid reservoir to the outlet via the first fluidic channel.

2. The handpiece device of claim 1, further comprising an angled portion coupling the tip portion to the shaft, the angled portion having a third fluidic channel in fluid communication with the first fluidic channel and the second fluidic channel.

3. The handpiece device of claim 2, wherein at least one of the angled portion or the tip portion are separable from the shaft, and can be coupled together using one or more of a snap-on connector, a friction fit connection, a press-fit connection, a knurled nut, or a Luer lock connection.

4. The handpiece device of claim 2, wherein at least one of the angled portion or the tip portion can rotate, while coupled to the shaft, around an axis parallel to a length of the shaft.

5. The handpiece device of claim 2, wherein the angled portion forms an angle between the shaft and the tip portion between 90 degrees and 170 degrees.

6. The handpiece device of claim 1, wherein the angled portion, the tip portion, and the shaft are manufactured from a single contiguous piece of material.

7. The handpiece device of claim 1, wherein the shaft or the tip portion are manufactured from one or more of stainless steel or a sterilisable plastic.

8. The handpiece device of claim 1, wherein the shaft comprises a first plurality of fluidic channels and the tip portion comprises a second plurality of fluidic channels, and wherein each of the second plurality of fluidic channels are in fluidic communication with a respective one of the first plurality of fluidic channels.

9. The handpiece device of claim 1, wherein the outlet of the tip portion further comprises a needle portion extending past the collar portion and configured to pierce an anatomic structure.

10. The handpiece device of claim 9, wherein the needle portion extends past the collar portion by a distance between 1 millimeter and 4 millimeters, or by a distance between 2 millimeters and 3 millimeters, and wherein the needle portion has a gauge size between 25 and 30.

11. The handpiece device of claim 1, wherein the shaft portion has a diameter of 4 millimeters, 5 millimeters, or 6 millimeters, and a length between 90 millimeters and 160 millimeters.

12. The handpiece device of claim 1, wherein the outlet of the tip portion is positioned at a distal end of the tip portion, the distal end forming an angle between the outlet and the tip portion, wherein the angle is between 70 degrees and 170 degrees, between 75 degrees and 130 degrees, between 90 degrees and 120 degrees, or between 110 degrees and 120 degrees.

13. The handpiece device of claim 1, wherein the micropump integrated with the shaft comprises the fluid reservoir, an electromagnetic actuator, a battery, and a control circuit.

14. A system, comprising:
a shaft of a handpiece device defining a channel;
a micropump integrated with the shaft of the handpiece device, the micropump comprising:
a fluid reservoir having a reservoir inlet and a reservoir outlet;
a fluidic channel fluidly coupled to the reservoir outlet of the fluid reservoir;
a pump comprising an electromagnetic actuator, the pump fluidly connected to the reservoir outlet of the fluid reservoir via the fluidic channel; and
an intake valve coupled to the fluidic channel between the fluid reservoir and the pump,
wherein the pump, when actuated, causes fluid in the fluid reservoir to flow to an outlet that is fluidly connected to the channel defined by the shaft of the handpiece; and
a tip portion coupled to the shaft of the handpiece, the tip portion having a second channel that receives fluid from the micropump via the channel defined by the shaft.

15. The system of claim 14, wherein the micropump comprises a plurality of layers, each of the plurality of layers defining at least one of the fluid reservoir, the fluidic channels, or the outlet.

16. The system of claim 14, wherein the outlet of the micropump is fluidly connected to the reservoir inlet of the fluid reservoir via a second valve.

17. The system of claim 14, wherein the micropump further comprises one or more fluid capacitors disposed between the fluid reservoir and the pump, or between the pump and the outlet.

18. A method comprising:
providing a handpiece device comprising:
a shaft comprising a first fluidic channel;
a tip portion coupled with a first end of the shaft and comprising an outlet and a second fluidic channel in fluid communication with the first fluidic channel; and
a collar coupled with the tip portion a predetermined distance from the outlet, the collar configured to seat with an anatomic structure and control a distance that the tip portion can project into a cochlea when the tip portion is inserted into an ear canal,
wherein the shaft is integrated with micropump and a fluid reservoir, the micropump configured to pump a fluid from the fluid reservoir to the outlet via the first fluidic channel,
piercing an anatomic membrane covering the anatomic structure of a patient with the tip portion of the handpiece device; and
flowing a fluid, using the micropump, through the outlet and into cochlea via the first fluidic channel and the second fluidic channel.

19. The method of claim 18, wherein piercing the anatomic membrane of the patient comprises:
inserting the tip portion of the handpiece through an ear canal of the patient;
pressing the tip portion through the anatomic membrane in a middle ear of the patient; and
seating the collar with the anatomic structure covered by the anatomic membrane in the middle ear, causing a part of the tip portion to extend into the cochlea of the patient.

20. The method of claim 18, further comprising forming a ventilation hole in a stapes footplate of the patient.
